(19) United States
(12) Patent Application Publication
(10) Pub. No.: US 2018/0140770 A1
(43) Pub. Date: May 24, 2018

(54) INFUSATE TUBING CLAMP SYSTEMS FOR INFUSION PUMPS

(71) Applicant: Smiths Medical ASD, Inc., Plymouth, MN (US)

(72) Inventors: Clint HETCHLER, Minnetonka, MN (US); Grant ADAMS, Anoka, MN (US); Chris LACY, Arden Hills, MN (US)

(73) Assignee: Smiths Medical ASD, Inc., Plymouth, MN (US)

(21) Appl. No.: 15/574,786

(22) PCT Filed: May 25, 2016
(86) PCT No.: PCT/US16/34015

§ 371 Date: Nov. 16, 2017

Related U.S. Application Data
(60) Provisional application No. 62/169,115, filed on Jun. 1, 2015.

(51) Int. Cl. A61M 5/168; A61M 5/145; A61M 39/28

(52) U.S. Cl. CPC A61M 5/16813; A61M 5/1452; A61M 39/281; A61M 5/16831; A61M 2205/3379; A61M 2205/702; A61M 2005/16863; A61M 2205/15; A61M 2205/33

(57) ABSTRACT

An infusate tubing clamp system for an infusion pump includes at least one movable tubing holder for removably securing a portion of infusate tubing. An actuator is operatively coupled to the at least one movable tubing holder. When the actuator is activated, the at least one movable tubing holder responsively and reversibly moves to a position that compressively clamps the portion of infusate tubing with sufficient force to temporarily and reversibly occlude the infusate tubing.

### TECHNICAL FIELD

Subject matter hereof relates generally to infusion pumps, and more particularly, to infusate tubing clamp systems for infusion pumps.

### BACKGROUND

Infusion pumps are useful medical devices for managing the delivery and dispensation of many types of therapeutic infusates. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large volume, patient-controlled analgesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an intraoperative site, epidural space or subarachnoid space.

Typically, when deploying an infusion pump for initial use with a particular patient or during various other procedures such as, for example, replacing a syringe in the pump, a hemostat or other separate mechanical clamping device can be employed to clamp or otherwise pinch and occlude the infusate tubing in a portion where the infusate exits the pump and leads to the patient. This separate task is typically performed so that, for example, infusate in the tubing does not leak out of tubing not yet connected to a patient or cause an unintended bolus delivery of infusate to a patient who is connected to the tubing. Such manual and rather disruptive, inefficient, and cumbersome tasks can cause difficulties in infusion protocols due to, for example, time needed to find hemostats or other clamping devices, manipulate them for placement on and occlusion of proper portions of infusate tubing, and then remove them after the intended occlusions are no longer needed. Furthermore, if the hemostat or other separate mechanical clamping device is inadvertently not removed after starting the infusion pump, an alarm may be triggered causing disruption and inefficiency in the patient's treatment or other procedure involving the pump as well.

Therefore it would be advantageous to provide integrated clamping and occlusion functionality with infusion pumps, to obviate a need for hemostats or other separate mechanical clamping devices and thereby minimize disruptions and inefficiencies in infusion protocols.

### SUMMARY

Embodiments described or otherwise contemplated herein substantially meet the aforementioned needs. For example, an infusate tubing clamp system for an infusion pump includes at least one movable tubing holder for removably securing a portion of infusate tubing. An actuator is operatively coupled to the at least one movable tubing holder. When the actuator is activated, the at least one movable tubing holder responsively and reversibly moves to a position that compressively clamps the portion of infusate tubing with sufficient force to temporarily and reversibly occlude the infusate tubing. In a feature and advantage of embodiments, the infusate tubing clamp system includes an infusion pump that is selected from a group consisting of a syringe pump, an ambulatory pump, a large volume pump, a peristaltic pump, and an elastomeric pump. In a feature and advantage of embodiments, the actuator is an electromechanical solenoid.

In an embodiment, an infusate tubing clamp system for an infusion pump includes at least one movable tubing holder for removably securing a portion of infusate tubing. An actuator is operatively coupled to the at least one movable tubing holder. An accelerometer is operatively coupled to the actuator. When the actuator is activated in response to a signal from the accelerometer, the at least one movable tubing holder responsively and reversibly moves to a position that compressively clamps the portion of infusate tubing with sufficient force to temporarily and reversibly occlude the infusate tubing. In a feature and advantage of embodiments, the infusate tubing clamp system includes an infusion pump that is selected from a group consisting of a syringe pump, an ambulatory pump, a large volume pump, a peristaltic pump, and an elastomeric pump. In a feature and advantage of embodiments, the actuator is an electromechanical solenoid.

In an embodiment, an infusate tubing clamp system for an infusion pump includes at least one tubing holder for removably securing a portion of infusate tubing. A tubing post is provided proximate to the at least one tubing holder. A rotatable clamp post is provided proximate to the tubing post; and an actuator is operatively coupled to the rotatable clamp post. When the actuator is activated, the rotatable clamp post responsively and reversibly rotates to a position that compressively clamps the portion of infusate tubing against the tubing post with sufficient force to temporarily and reversibly occlude the infusate tubing. In a feature and advantage of embodiments, the infusate tubing clamp system includes an infusion pump that is selected from a group consisting of a syringe pump, an ambulatory pump, a large volume pump, a peristaltic pump, and an elastomeric pump. In a feature and advantage of embodiments, the actuator is an electromechanical solenoid.

In an embodiment, an infusate tubing clamp system for an infusion pump includes at least one tubing holder for removably securing a portion of infusate tubing. A tubing post is provided proximate to the at least one tubing holder. A rotatable clamp post is provided proximate to the tubing post. An actuator is operatively coupled to the rotatable clamp post; and an accelerometer is operatively coupled to the actuator. When the actuator is activated in response to a signal from the accelerometer, the rotatable clamp post responsively and reversibly rotates to a position that compressively clamps the portion of infusate tubing against the tubing post with sufficient force to temporarily and reversibly occlude the infusate tubing. In a feature and advantage of embodiments, the infusate tubing clamp system includes an infusion pump that is selected from a group consisting of a syringe pump, an ambulatory pump, a large volume pump, a peristaltic pump, and an elastomeric pump. In a feature and advantage of embodiments, the actuator is an electromechanical solenoid.

In an embodiment, an infusate tubing clamp system for an infusion pump includes a first tubing guide member and a second tubing guide member, for removably guiding a portion of infusate tubing. The first tubing guide member is movable toward the second tubing guide member; and an actuator is operatively coupled to the first tubing guide member. When the actuator is activated, the first tubing guide member responsively and reversibly moves to a position that compressively clamps the portion of infusate tubing against the second tubing guide member with sufficient force to temporarily and reversibly occlude the infusate tubing. In a feature and advantage of embodiments, the infusate tubing clamp system includes an infusion pump that is selected from a group consisting of a syringe pump, an ambulatory pump, a large volume pump, a peristaltic pump, and an elastomeric pump. In a feature and advantage of embodiments, the actuator is an electromechanical solenoid.

In an embodiment, operation of an infusion pump includes an infusate tubing clamp system for the infusion pump. A method of the operation is selected from a group of preventing “crosstalk”, improving startup performance, running a pump motor in reverse to pull backwardly on a syringe plunger and thereby mitigate any unintended bolus of infusate that would otherwise be delivered from the syringe, providing a test of the pump motor, providing a test of a downstream occlusion sensor, providing a test of motor health, providing a test of motor rate error prevention, determining a presence and amount of air in infusate tubing, determining whether there may be a leak or a misconnection somewhere in the infusate's flow path, estimating an internal diameter of a syringe, and estimating a fluid volume capacity of a syringe.

Regardless of a particular infusate tubing clamp system as described by example or otherwise contemplated herein, embodiments herein are not limited to the examples explicitly provided. The above summary is not necessarily intended to describe each illustrated embodiment or every implementation of the subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments of the subject matter in connection with the accompanying drawings, in which:

FIG. 1 is a front view of a syringe type infusion pump.

FIG. 2 is a perspective view of an example of an infusate tubing clamp system for an infusion pump, according to an embodiment.

FIG. 2A is a perspective view of the infusate tubing clamp system for an infusion pump of FIG. 2, depicting activation of the system.

FIG. 3 is a perspective view of another example of an infusate tubing clamp system for an infusion pump, according to an embodiment.

FIG. 3A is a top view of the infusate tubing clamp system for an infusion pump of FIG. 3.

FIG. 3B is a perspective view of the infusate tubing clamp system for an infusion pump of FIG. 3, depicting activation of the system.

FIG. 3C is a top view of the infusate tubing clamp system for an infusion pump of FIG. 3B.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION OF THE DRAWINGS

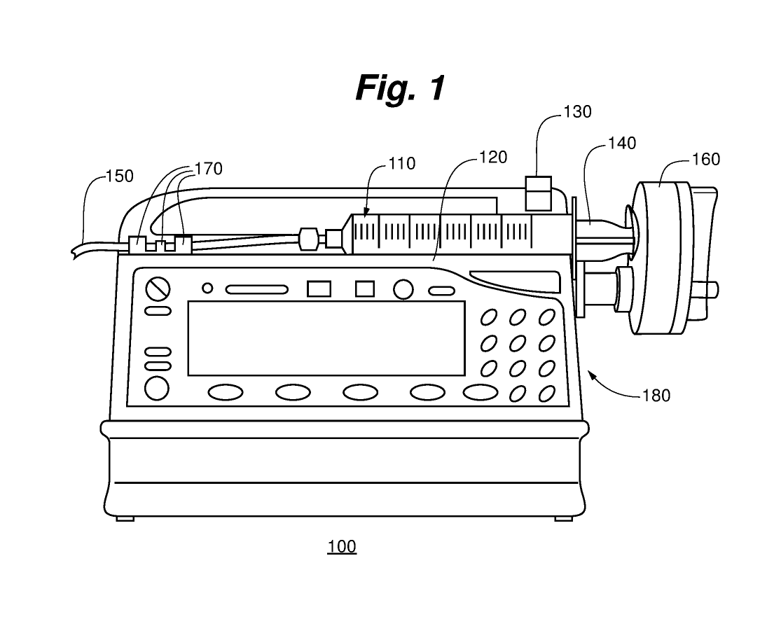

FIG. 1 illustrates an infusion pump 100, such as, for example, a MEDFUSION 4000 infusion pump from Smiths Medical ASD, Inc. In general, infusion pump 100 is a syringe-type pump that can be used to deliver various infusates, drug therapies and treatments to patients. When in use, infusion pump 100 typically includes a removable and replaceable pharmaceutical container or syringe 110, which is supported on and secured to housing 120 of pump 100 and can be secured thereto by clamp 130. In embodiments, syringe 110 can be separately supplied from pump 100. In other embodiments, syringe 110 can be an integrated component of pump 100. Syringe 110 includes a plunger 140 that forces fluid outwardly from syringe 110 via infusate tubing 150 that is connected to a patient (not illustrated). A pusher or plunger driver mechanism 160, when in operation, acts to move plunger 140 of syringe 110. Operation of mechanism 160 can be provided by way of, for example, cooperative action of a motor and lead screw arrangement internal to housing 120 of pump 100. In embodiments, a sensor (not shown; which is typically internal to plunger driver mechanism 160) monitors force and/or plunger position in the syringe according to system specifications. Pump 100 also typically includes tubing holders 170, for guiding and removably securing a portion of infusate tubing 150 to housing 120 of pump 100. Pump 100 further typically includes a user interface 180 with an integrated display screen for relaying commands to a control system (not illustrated) of pump 100. User interface 180 generally allows a user to enter various parameters, including but not limited to names, drug information, limits, delivery shapes, information relating to hospital facilities, as well as various user-specific parameters (e.g., patient age and/or weight). Infusion pump 100 can include a USB port or other appropriate input/output (I/O) interface port (not illustrated) for connecting pump 100 to a network or computer (not illustrated) having software designed to interface with pump 100. Power to infusion pump 100 is accomplished via an AC or DC power cord or an internally provided battery source (not illustrated). Embodiments can also include a wireless power source (not illustrated). User inputs to infusion pump 100 can be provided by programming from a user, such as a patient, pharmacist, scientist, drug program designer, medical engineer, nurse, physician, or other medical practitioner or healthcare provider. User inputs may utilize direct interfacing (via, e.g., keyboards, touch screens, or other touch-based inputs) as shown, or user inputs may utilize indirect or “touchless” interfacing (i.e., gestures; voice commands; facial movements or expressions; finger, hand, head, body and arm movements; or other inputs that do not require physical contact such as cameras, sensors of electric field, capacitance, or sound). User inputs are generally interfaced, communicated, sensed, and/or received by operator input mechanisms of user interface 180.

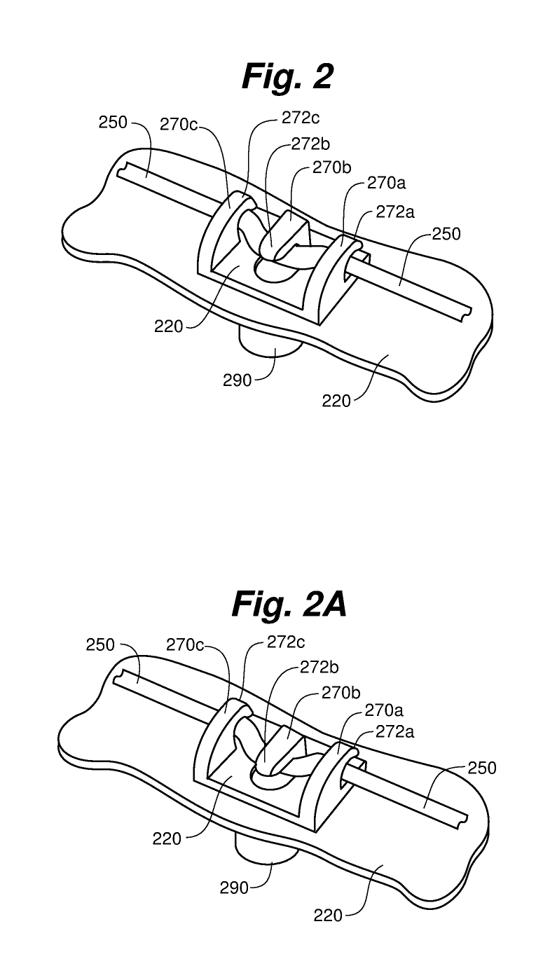

FIG. 2 is a perspective view of an example of an infusate tubing clamp system for an infusion pump, according to an embodiment. In particular in FIG. 2, with reference to housing 120 and holders 170 of FIG. 1, a portion of an infusion pump housing 220 is shown in a magnified view, with tubing holders 270a, 270b, and 270c. As illustrated, tubing holders 270a-c function to guide and removably secure a portion of infusate tubing 250 to housing 220. It is to be understood that, as shown in FIG. 2, tubing 250 has been threaded through or otherwise placed under arch-like portions 272a, 272b, and 272c of holders 270a-c, respectively, by a user of the pump. In the example of FIG. 2, tubing holders 270a and 270c are fixed to or stationary on housing 220; but tubing holder 270b is moveable vertically, upwardly and downwardly in the drawing. Such upward and downward vertical motion of moveable tubing holder 270b is provided by an actuator 290 in housing 220 that is operatively coupled to movable tubing holder 270b. In an embodiment, actuator 290 is an electromechanical solenoid. In this configuration, and with reference also to FIG. 2A, when actuator 290 is activated to intentionally occlude infusate tubing 250 as will be further described, movable tubing holder 270b responsively moves to a position (downwardly in the drawing) that compressively clamps tubing 250 against infusion pump housing 220 with sufficient force to temporarily occlude tubing 250.

Actuation of moveable tubing holder 270b may be desired when, for example, a syringe is being replaced in the pump. In an embodiment, such actuation may be initiated by a user of the pump via a suitable control interface (such as, e.g., a user interface 180 as shown in FIG. 1). In response, a control system of the pump can command actuator 290 to drive moveable tubing holder 270b downwardly as aforedescribed. In another embodiment, an accelerometer (not illustrated) can be operatively coupled to actuator 290. The accelerometer can be configured to transmit an actuation signal to actuator 290 (or, via a microprocessor or microcontroller in communication with actuator 290) upon, for example, sensing an acceleration that would be indicative of an unintended physical occurrence at the pump such as a sudden movement or impact which could potentially cause a disconnection of the infusate tubing from the pump, a leak from tubing not yet connected to a patient, or an unintended bolus delivery of infusate to a patient who is connected to the tubing. The accelerometer can also be configured to transmit an actuation signal to actuator 290 (or, via a microprocessor or microcontroller in communication with actuator 290) upon, for example, sensing an acceleration that would be indicative of any other unintended physical occurrence at the pump such as during patient transport in land vehicles across rough terrain or in aircraft through turbulent air. In these difficult environments, the infusate tubing clamp system could prevent deleterious “freeflow” in circumstances where, for example, a syringe or other infusate reservoir becomes dislodged from an infusion pump. Upon sensing such accelerations, therefore, actuator 290 would then drive moveable tubing holder 270b downwardly as aforedescribed to a position that compressively clamps infusate tubing 250 against infusion pump housing 220 with sufficient force to temporarily occlude tubing 250. Accordingly, then, the potential disconnection, leak, or unintended bolus could be prevented.

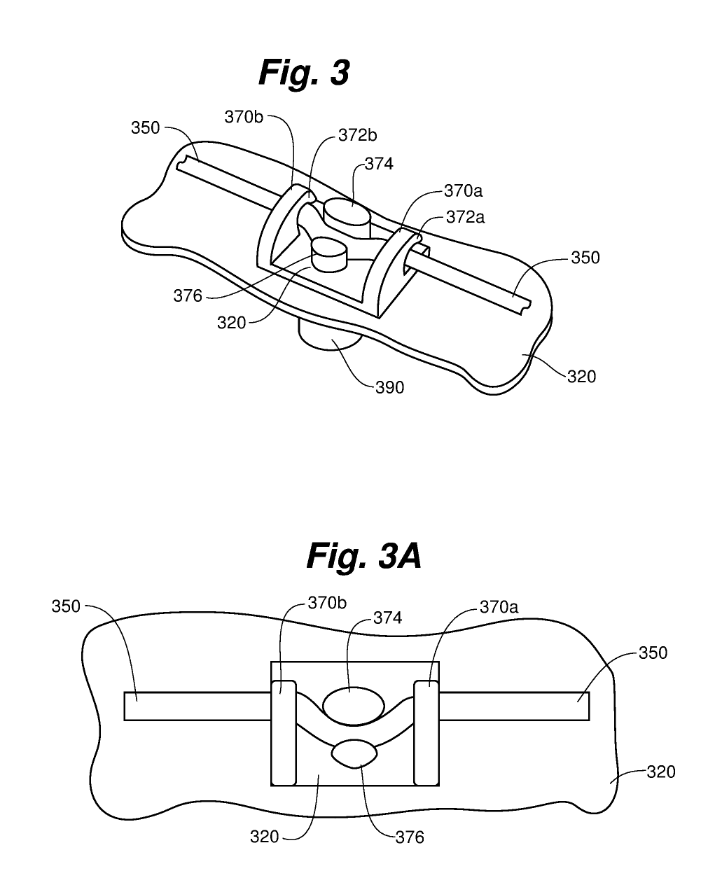

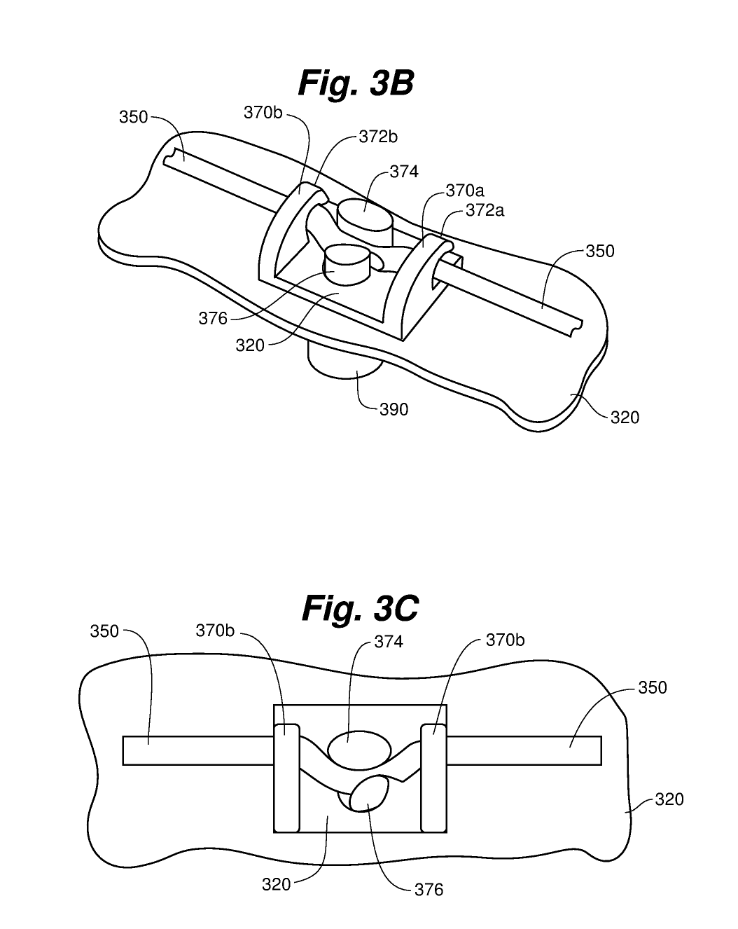

It is to be appreciated and understood that actuator 290 can also reversibly drive tubing holder 270b, to remove compressive force from tubing 250 (upwardly in FIG. 3). Such movement of holder 270b may be desired when, for example, the syringe has been replaced, the pump is ready to resume an infusion operation or protocol, and tubing 250 may therefore be safely un-occluded and re-opened to provide delivery of infusate from the pump to the patient.

FIG. 3 is a perspective view of another example of an infusate tubing clamp system for an infusion pump, according to an embodiment. In particular in FIG. 3, and with reference also to FIG. 3A and housing 120 and holders 170 of FIG. 1, a portion of an infusion pump housing 320 is shown in a magnified view, with tubing holders 370a and 370b. As illustrated, tubing holders 370a-b function to guide and removably secure a portion of infusate tubing 350 to housing 320. It is to be understood that, as shown in FIG. 3 and FIG. 3A, tubing 350 has been threaded through or otherwise placed under arch-like portions 372a and 372b of holders 370a-b, respectively, by a user of the pump. In the example of FIG. 3, tubing holders 370a-b are fixed to or stationary on housing 320. A tubing post 374 is also fixed to or stationary on housing 320, proximate to tubing holders 370a-b. A rotatable clamp post 376 on housing 320 is proximate to tubing post 374. Rotatable clamp post 376 is rotatable clockwise and counter-clockwise about its vertical axis in the drawings (as shown in comparing, for example, FIG. 3A to FIG. 3C). Such rotating motion of rotatable clamp post 376 is provided by an actuator 390 in housing 320 that is operatively coupled to rotatable clamp post 376. In an embodiment, actuator 390 is an electromechanical solenoid. In this configuration, when actuator 390 is activated to intentionally occlude infusate tubing 350 as will be further described, rotatable clamp post 376 responsively and rotates to a position (counter-clockwise in FIG. 3B and FIG. 3C, relative to FIG. 3 and FIG. 3A, respectively) that compressively clamps tubing 350 against tubing post 374 with sufficient force to temporarily occlude tubing 350.

Actuation of rotatable clamp post 376 may be desired when, for example, a syringe is being replaced in the pump. In an embodiment, such actuation may be initiated by a user of the pump via a suitable control interface (such as, e.g., a user interface 180 as shown in FIG. 1). In response, a control system of the pump can command actuator 390 to drive rotatable clamp post 376 counter-clockwise as aforedescribed. In another embodiment, an accelerometer (not illustrated) can be operatively coupled to actuator 390. The accelerometer can be configured to transmit an actuation signal to actuator 390 (or, via a microprocessor or microcontroller in communication with actuator 390) upon, for example, sensing acceleration as aforedescribed with reference to the example embodiment of FIG. 2. Upon sensing such accelerations, therefore, actuator 390 would then drive rotatable clamp post 376 counter-clockwise as aforedescribed to a position that compressively clamps infusate tubing 350 against tubing post 374 with sufficient force to temporarily occlude tubing 350. Accordingly, then, the potential disconnection, leak, or unintended bolus could be prevented.

It is to be appreciated and understood that actuator 390 can also reversibly drive rotatable clamp post 376, to remove compressive force from tubing 350 (clockwise with reference to FIG. 3B and FIG. 3C) and return to a relaxed state as shown in FIG. 3 and FIG. 3A, respectively. Such movement of rotatable clamp post 376 may be desired when, for example, the syringe has been replaced, the pump is ready to resume an infusion operation or protocol, and tubing 350 may therefore be safely un-occluded and re-opened to provide delivery of infusate from the pump to the patient.

In another embodiment, although not illustrated, an infusate tubing clamp system for an infusion pump includes two infusate tubing guide members which face each other. The tubing guide members can be characterized as being similar to holders 270 and 370 of FIGS. 2-3C. However, these tubing guide members face each other and omit arch-like portions 272 and 372 of holders 270 and 370; and one or both of them would be capable of responsive movement relative to each other. For example, in an embodiment of two such tubing guide members, a first tubing guide member is movable in a slot in the pump housing toward a second tubing guide member that is fixed to or stationary on the housing. The motion of the first tubing guide member is provided by an actuator in the housing that is operatively coupled to the first tubing guide member. In an embodiment, the actuator is an electromechanical solenoid. In this configuration, when the actuator is activated to intentionally occlude a portion of infusate tubing as will be further described, the first tubing guide member responsively moves toward the second tubing guide member. Such movement of the first tubing guide member toward the second tubing guide member thereby compressively clamps the tubing against the second tubing guide member with sufficient force to temporarily occlude the tubing.

Actuation of the first tubing guide member may be desired when, for example, a syringe is being replaced in the pump as aforedescribed relative to the example embodiments of FIGS. 2 and 3. Also as aforedescribed, an accelerometer can be operatively coupled to the actuator and configured to transmit an actuation signal to the actuator (or, via a microprocessor or microcontroller in communication with the actuator) upon, for example, sensing an acceleration as aforedescribed with reference to the example embodiments of FIGS. 2 and 3. Upon sensing such accelerations, therefore, the actuator then drives the first tubing guide member toward the second tubing guide member and to a position that compressively clamps the infusate tubing against the second tubing guide member with sufficient force to temporarily occlude the tubing. Accordingly, then, the potential disconnection, leak, or unintended bolus could be prevented.

Also as aforedescribed with reference to the example embodiments of FIGS. 2 and 3, it is to be appreciated and understood that the actuator can also reversibly drive the first tubing guide member away from the second tubing guide member, to remove compressive force from the tubing. Such movement of the first tubing guide member may be desired when, for example, the syringe has been replaced, the pump is ready to resume an infusion operation or protocol, and the tubing may therefore be safely un-occluded and re-opened to provide delivery of infusate from the pump to the patient.

Although not specifically illustrated herein, it is also to be appreciated and understood that infusate tubing clamp systems for infusion pumps—as described by example or otherwise contemplated herein—could be useful for prevention of so-called “crosstalk” between separate infusates being delivered to a patient. For example, when two drugs are highly incompatible but are both being delivered to a patient in a “piggybacking” infusion protocol, there may be a risk of migration of one drug to the other. To mitigate this risk an infusate tubing clamp system could be activated manually, or even automatically by the system upon potential occurrence of such crosstalk by way of a suitable sensor and control technique (not illustrated).

Also although not specifically illustrated herein, it is also to be appreciated and understood that infusate tubing clamp systems for infusion pumps—as described by example or otherwise contemplated herein—could be useful for improving startup performance. In such an embodiment, an infusate tubing clamp system could be activated with the syringe plunger driven by the pump until a known pressure is achieved. The clamp system could then be released therefore starting the infusion with no or minimal delay. For example, in an embodiment of improved startup performance provided by an infusate tubing clamp system for an infusion pump, the pump would use its occlusion pressure sensor to detect a first pressure. The infusate tubing clamp system would then be activated, to temporarily occlude the tubing. The pump's motor would then be run to advance the syringe plunger until the pump's occlusion pressure sensor detects a second pressure that is of a selected higher pressure than the first pressure. A suitable microprocessor would be employed by or in the pump, to calculate the second pressure for a particular use of improved startup performance provided by the infusate tubing clamp system. In an embodiment, such calculation could take into account selected physical parameters such as certain syringe and/or tubing characteristics and thereby infer that the syringe plunger has been advanced far enough to effectively remove mechanical “slack”, “play”, or “backlash” from the drive train of the pump. In an embodiment, it could be possible to simply infer with reference to a pressure sensor coupled to the syringe or tubing that such slack, play, or backlash has been satisfactorily accommodated. As such, irrespective of a particular embodiment, force placed on the syringe plunger could be advantageously increased in a significantly shorter amount of time than if the motor simply ran (i) at its intended rate, as described in WIPO Applic. No. PCT/US2015/013049, filed on 27 Jan. 2015, and titled “Pump Startup Algorithms and Related Systems and Methods” (with the disclosure of this PCT application being incorporated herein, by reference thereto) or perhaps (ii) at a nominal rate for a predetermined time interval without benefit of pressure feedback information. After a selected time following occurrence of sensing the second pressure, the motor would then be stopped and the infusate tubing clamp system would be de-activated/opened, to remove the temporary occlusion of the tubing. In an embodiment, the selected time and the actions of stopping the motor and de-activating/opening the clamp would be controlled by the aforementioned microprocessor. It is also to be appreciated and understood that in an embodiment of an infusate tubing clamp system for an infusion pump, the system could command the pump's motor to run in reverse to pull backwardly on the plunger and thereby mitigate, reduce, or eliminate any unintended bolus of infusate that would otherwise be delivered to the patient due to, for example, a pressure that exceeded an optimal pressure for improved startup performance.

Also although not specifically illustrated herein, it is also to be appreciated and understood that infusate tubing clamp systems for infusion pumps—as described by example or otherwise contemplated herein—could provide several other internal features in addition to the aforedescribed user-facing features. For example, clamping the infusate tubing and increasing pressure within the tubing by activating the pump's motor to drive the pump could be used as a self-test of both the pump's motor and downstream occlusion sensor. Increasing the pressure even further could be used as a test of motor health or motor rate error prevention. Furthermore, a suitable embodiment of an infusate tubing clamp system could be used to determine a presence and amount of air in the infusate tubing. In this regard, the amount of air present in the infusate tubing could be roughly calculated by clamping the tubing and measuring how far the syringe has to travel before a specified pressure is reached. For example, in an embodiment of air detection provided by an infusate tubing clamp system for an infusion pump, the system could function analogously to the aforedescribed improved startup performance feature. In such an embodiment of air detection, the pump would use its occlusion pressure sensor to detect a first pressure. The infusate tubing clamp system would then be activated, to temporarily occlude the tubing. The pump's motor would then be run to advance the pump's syringe plunger driver until the occlusion pressure sensor detects a second pressure that is of a selected higher pressure than the first pressure. A suitable microprocessor would be employed by or in the pump, to calculate the second pressure versus forward displacement of the pump's syringe plunger driver for a particular use of air detection provided by the infusate tubing clamp system. In an embodiment, such calculation could take into account selected physical parameters such as certain syringe and/or tubing characteristics. A forward displacement of the syringe plunger driver that does not result in an increase of pressure to the second pressure that was expected or predicted by the microprocessor would thereby result in a conclusion that air may be present in the tubing, and/or there may be a leak or a misconnection somewhere in the infusate's flow path.

Also although not specifically illustrated herein, it is also to be appreciated and understood that infusate tubing clamp systems for infusion pumps—as described by example or otherwise contemplated herein—could provide yet another feature. For example, clamping the infusate tubing and increasing pressure within the syringe by activating the pump could be used to estimate the syringe's internal diameter and fluid volume capacity. Such estimate of the internal diameter could be used to reduce a number of possible syringes used in the pump, or to double-check or aid in verifying that a correct syringe has been selected for use in the pump. Such a feature could provide greater infusion safety by minimizing a chance of delivering an incorrect amount of medication to the patient. In this regard, a particular syringe pump may only be able to measure an external diameter of a syringe based on, e.g., travel or displacement of a syringe barrel holder or clamp in the pump when the syringe is installed in the pump; and various types and sizes of syringes may have similar diameters but dissimilar internal diameters. For example, some 3 mL and 1 mL syringes have similar external diameters but significantly different internal diameters. In an embodiment of this feature of syringe internal diameter detection provided by an infusate tubing clamp system as described by example or otherwise contemplated herein, the system could function analogously to the aforedescribed improved startup performance feature. In particular, the pump could use its occlusion pressure sensor to detect a first pressure. The infusate tubing clamp system could then be activated, to temporarily occlude the tubing. The pump's motor could then be run to advance the pump's syringe plunger driver until the occlusion pressure sensor detects a second pressure that is of a selected higher pressure than the first pressure. A suitable microprocessor could be employed by or in the pump, to calculate the second pressure versus forward displacement of the pump's syringe plunger driver for a particular syringe to approximately determine the internal diameter of the syringe. In an embodiment, such calculation could take into account selected physical parameters such as distance of travel of the plunger driver relative to the sensed occlusion pressure. A forward displacement of the syringe plunger driver that does not result in an increase of pressure to the second pressure that was expected or predicted by the microprocessor relative to selected physical syringe characteristics (e.g., outer diameter, length, etc.) could thereby result in a conclusion or alarm that an incorrect syringe may have been selected and installed in the pump.

Although described with particular reference to syringe pumps, it is to be appreciated and understood that the novel and inventive infusate tubing clamp systems that have been described by example or are otherwise contemplated herein may also be used with any suitable infusion pumps (such as, for example, so-called ambulatory pumps, large volume pumps, peristaltic pumps, and elastomeric pumps, etc.) provided that suitable components and systems thereof satisfactorily function in cooperation with the infusate tubing clamp systems according to subject matter hereof.

Various embodiments of systems, devices, and methods have been described herein. These embodiments are given only by way of example and are not intended to limit the scope of subject matter hereof. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized commensurate with the scope of subject matter hereof.

Persons of ordinary skill in the relevant arts will recognize that subject matter hereof may comprise fewer features than illustrated in any individual embodiment described above. The embodiments described herein are not meant to be an exhaustive presentation of the ways in which the various features of subject matter hereof may be combined. Accordingly, the embodiments are not mutually exclusive combinations of features; rather, the subject matter hereof may comprise a combination of different individual features selected from different individual embodiments, as understood by persons of ordinary skill in the art. Thus, for example, it is to be appreciated and understood that a particular embodiment of subject matter hereof could have any number, more or fewer, of tubing holders 270a and 270c, and 370a and 370b, than shown in the example embodiments of FIGS. 2 and 3, respectively; and a particular embodiment of subject matter hereof could have more than one movable tubing holder 270b, and tubing post 374 and rotatable clamp post 376, as well.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims of subject matter hereof, it is expressly intended that the provisions of Section 112, sixth paragraph of 35 U.S.C. are not to be invoked unless the specific terms “means for” or “step for” are recited in a claim.

1. An infusate tubing clamp system for an infusion pump, comprising:
at least one movable tubing holder for removably securing a portion of infusate tubing; and
an actuator operatively coupled to the at least one movable tubing holder,
wherein when the actuator is activated, the at least one movable tubing holder responsively and reversibly moves to a position that compressively clamps the portion of infusate tubing with sufficient force to temporarily and reversibly occlude the infusate tubing.

2. The infusate tubing clamp system for an infusion pump of claim 1, wherein the infusion pump is selected from a group consisting of a syringe pump, an ambulatory pump, a large volume pump, a peristaltic pump, and an elastomeric pump.

3. The infusate tubing clamp system for an infusion pump of claim 1, wherein the actuator is an electromechanical solenoid.

4. An infusate tubing clamp system for an infusion pump, comprising:
at least one movable tubing holder for removably securing a portion of infusate tubing;
an actuator operatively coupled to the at least one movable tubing holder; and
an accelerometer operatively coupled to the actuator,
wherein when the actuator is activated in response to a signal from the accelerometer, the at least one movable tubing holder responsively and reversibly moves to a position that compressively clamps the portion of infusate tubing with sufficient force to temporarily and reversibly occlude the infusate tubing.

5. The infusate tubing clamp system for an infusion pump of claim 4, wherein the infusion pump is selected from a group consisting of a syringe pump, an ambulatory pump, a large volume pump, a peristaltic pump, and an elastomeric pump.

6. The infusate tubing clamp system for an infusion pump of claim 4, wherein the actuator is an electromechanical solenoid.

7. An infusate tubing clamp system for an infusion pump, comprising:
at least one tubing holder for removably securing a portion of infusate tubing;
a tubing post proximate to the at least one tubing holder;
a rotatable clamp post proximate to the tubing post; and
an actuator operatively coupled to the rotatable clamp post,
wherein when the actuator is activated, the rotatable clamp post responsively and
reversibly rotates to a position that compressively clamps the portion of infusate tubing against the tubing post with sufficient force to temporarily and reversibly occlude the infusate tubing.

8. The infusate tubing clamp system for an infusion pump of claim 7, wherein the infusion pump is selected from a group consisting of a syringe pump, an ambulatory pump, a large volume pump, a peristaltic pump, and an elastomeric pump.

9. The infusate tubing clamp system for an infusion pump of claim 7, wherein the actuator is an electromechanical solenoid.

10. An infusate tubing clamp system for an infusion pump, comprising:
at least one tubing holder for removably securing a portion of infusate tubing;
a tubing post proximate to the at least one tubing holder;
a rotatable clamp post proximate to the tubing post;
an actuator operatively coupled to the rotatable clamp post; and
an accelerometer operatively coupled to the actuator,
wherein when the actuator is activated in response to a signal from the accelerometer, the rotatable clamp post responsively and reversibly rotates to a position that compressively clamps the portion of infusate tubing against the tubing post with sufficient force to temporarily and reversibly occlude the infusate tubing.

11. The infusate tubing clamp system for an infusion pump of claim 10, wherein the infusion pump is selected from a group consisting of a syringe pump, an ambulatory pump, a large volume pump, a peristaltic pump, and an elastomeric pump.

12. The infusate tubing clamp system for an infusion pump of claim 10, wherein the actuator is an electromechanical solenoid.

13. An infusate tubing clamp system for an infusion pump, comprising:
a first tubing guide member and a second tubing guide member, for removably guiding a portion of infusate tubing, wherein the first tubing guide member is movable toward the second tubing guide member; and
an actuator operatively coupled to the first tubing guide member,
wherein when the actuator is activated, the first tubing guide member responsively and
reversibly moves to a position that compressively clamps the portion of infusate tubing against the second tubing guide member with sufficient force to temporarily and reversibly occlude the infusate tubing.

14. The infusate tubing clamp system for an infusion pump of claim 13, wherein the infusion pump is selected from a group consisting of a syringe pump, an ambulatory pump, a large volume pump, a peristaltic pump, and an elastomeric pump.

15. The infusate tubing clamp system for an infusion pump of claim 13, wherein the actuator is an electromechanical solenoid.

16. Operation of an infusion pump, including an infusate tubing clamp system for an infusion pump and a method of operation selected from a group as disclosed and described herein of:
preventing “crosstalk”;
improving startup performance;
running a pump motor in reverse to pull backwardly on a syringe plunger and thereby
mitigate any unintended bolus of infusate that would otherwise be delivered from the syringe;
providing a test of the pump motor;
providing a test of a downstream occlusion sensor;
providing a test of motor health;
providing a test of motor rate error prevention;
determining a presence and amount of air in infusate tubing;
determining whether there may be a leak or a misconnection somewhere in the infusate's flow path;
estimating an internal diameter of a syringe; and
estimating a fluid volume capacity of a syringe.
